(19) United States
(12) Patent Application Publication
(10) Pub. No.: US 2007/0179809 A1
(43) Pub. Date: Aug. 2, 2007

(54) System and method for performing a cost-utility analysis of pharmaceutical interventions

(76) Inventors: Melissa M. Brown, Flourtown, PA (US); Gary C. Brown, Flourtown, PA (US); Sanjay Sharma, Kingston (CA)

Correspondence Address: BLANK ROME LLP, 600 NEW HAMPSHIRE AVENUE, N.W., WASHINGTON, DC, 20037, US

(21) Appl. No.: 11/648,628

(22) Filed: Jan. 3, 2007

Related U.S. Application Data
(60) Provisional application No. 60/754,632, filed on Dec. 30, 2005.

(51) Int. Cl. G06Q 10/00; A61B 5/00

(52) U.S. Cl. 705/2; 600/300

(57) ABSTRACT

A system and a method for performing a cost-utility analysis of pharmaceutical interventions where each pharmaceutical intervention is associated with several potential health states. The system includes a processor and a database. The database contains for each pharmaceutical intervention several utility values associated with each health state and a probability for each potential health state associated with each pharmaceutical intervention. The processor is in communication with the database and determines a mean utility value for each pharmaceutical intervention by correlating each probability associated with each pharmaceutical intervention with the utility value associated with the respective health state. The processor also compares the pharmaceutical interventions the mean utility values of the pharmaceutical interventions by decision analysis.

### RELATED APPLICATION

This application claims priority to provisional application Ser. No. 60/754,632, filed Dec. 30, 2005, which is hereby incorporated by reference.

### BACKGROUND OF THE INVENTION

1. Field of the Invention

The present invention relates to a system and method for performing economic analysis of pharmaceutical interventions. More particularly, the present invention relates to a system and method for performing a cost-utility analysis of pharmaceutical interventions.

2. Background of the Related Art

As described in the publication Evidence-Based to Value-Based Medicine by Melissa M. Brown, Gary C. Brown & Sanjay Sharma (AMA Press 2005) at pages 27-46, the total healthcare expenditure in the United States is enormous and has been rising faster than the general rate of inflation over the last three decades of the 20th century. Although the United States spends a great deal of resources on healthcare, the allocation of healthcare resources has been inefficient. Resources are sometimes spent on healthcare interventions that provide negligible value, no value, or negative value because they are actually harmful. Once healthcare resources lost to allocation inefficiencies are identified, the recovered savings can be used to provide healthcare for the uninsured or those facing healthcare rationing.

To mitigate resources lost to inefficiencies, healthcare economic analyses have been increasingly used to evaluate healthcare interventions. As described in the Brown publication, pages 251-257, there are four basic forms of healthcare economic analysis: cost-minimization analysis, cost-benefit analysis, cost-effectiveness analysis, and cost-utility analysis.

Cost-minimization analysis essentially involves assessing two interventions of identical effectiveness to ascertain which one is less costly. It leads to healthcare decisions based solely upon minimizing costs. Cost-minimization analysis is rarely used because it ignores the fact that maximizing efficient use of healthcare resources can be achieved not only by spending less money but also by deriving the greatest value possible for the money expended. For example, cataract surgery is often accomplished in general surgical centers rather than in specialty ophthalmology surgical centers where costs are lower and value is often higher because of more readily available equipment and more personnel familiar with ophthalmologic procedures.

Cost-benefit analysis compares the costs expended on an intervention with the costs saved as a result of the intervention. This is done by measuring the amount of money that is saved by an intervention and the money that is expended. It is more readily understood than cost-effectiveness analysis or cost-utility analysis because cost-benefit analysis allows direct comparison of dollars expended to dollars saved. However, assigning a monetary value to a particular health condition is very difficult, controversial, and may only be possible in limited situations.

Cost-effectiveness analysis measures the costs expended for a particular outcome, such as the life-years gained, healthy years gained, years of good vision gained, and other similar outcomes. This is done by measuring the amount of money that is expended on an intervention for a particular outcome. The outcome is often measured in years of life, but years of life may not always be appropriate or useful. In particular, the quantity of life added is not accounted for in this analysis.

Cost-utility analysis measures cost expended for the value, such as improvement in the length of life and quality of life conferred by an intervention. This is done by measuring the amount of money expended on an intervention for the value gained. The value gained can be improvements in the length of life and/or the quality of life. The results of the analysis are dollars expended per quality-adjusted year of life gained. The cost-utility analysis is the most comprehensive form of healthcare economic analysis, but also the most complex. This analysis can transform evidence-based medicine to value-based medicine which allows resources to be shifted from interventions that have no value, negligible value, or harmful effects to interventions that work for all patients. Thus, cost-utility analysis has advantages over the other forms of healthcare economic analyses.

As explained in the Brown publication, pages 3-5, evidence-based medicine is the practice of medicine based upon the best scientific data available. It is a problem-solving approach that has been used for many years to gather information, process that information, and attempt to utilize the most important, relevant, and useful information.

Value-based medicine is the practice of medicine based upon patient-perceived value conferred by an intervention. Thus, value-based medicine evaluates the worth of a healthcare intervention to a patient. Also, value-based medicine allows patients to receive higher-quality care than evidence-based medicine alone since value-based medicine takes into account the patient's perception and not merely the clinical test results. Further, resources are better channeled so that economic resources that can be used to pay for healthcare services for those who are currently uninsured or experiencing rationing.

Value-based medicine principles are highly applicable to the sciences of pharmacoeconomics, as explained in the Brown publication, pages 301-302. Value-based medicine has played a minor role to date in regard to the utilization of pharmaceutical interventions because of: (1) lack of healthcare economic competence among formulary members, (2) an inadequate supply of relevant, value-based studies, (3) difficulty in translating the results of cost-utility studies into clinical guidelines, and (4) a lack of cost-utility analysis standards. The cost-utility analysis can help pharmaceutical manufacturers evaluate new drugs for development and drugs being evaluated for FDA approval. Value-based medicine allows pharmaceutical manufacturers to readily demonstrate the value of their drugs to those in healthcare. It identifies drugs of the same or greater value for less cost. Thus, value-based medicine will allow pharmaceutical dollars to be spent in the most efficient manner and facilitate the provision of pharmaceuticals to all patients in need.

The lack of standards for cost-utility analysis prevents widespread acceptance of value-based medicine, as noted in the Brown publication, pages 15-16. Current standards for cost-utility analysis are arbitrary at best. Cost-utility analysis requires utility values. Utility values vary with a health state of the patient which is the state of a person's health and can range from death to perfect health. So, utility values can vary from a lower value of zero (0.0) equated with death or another reference health state or to an upper value of one (1.0) representing permanent perfect health. The closer the utility value is to 0.0, the poorer the quality of life associated with the health state, and the closer the utility value is to 1.0, the better the quality of life associated with the health state. However, a perfectly healthy patient can have less than 1.0 for his utility value. Concerns about the future can cause a symptom less perfectly healthy patient to have an associated utility value less than 1.0. Large utility value decrements correlate to critically important function losses, such as loss of occupation, loss of the ability to walk, loss of the ability to read, and other similar losses.

Utility values are obtained by asking patients about their health state and correlating their responses to a scale from 0.0 to 1.0. Enough patients should be asked about a particular health state so that the utility value obtained for that health state can be statistically applied to another group of patients with the same health state.

Utility values are derived from quality-of-life measurement instruments. Several different quality-of-life instruments are available, such as a gambling utility analysis, willingness-to-pay utility analysis, time-tradeoff utility analysis, continuous scaling instruments, multiattribute instruments, and other similar utility analyses. However, there are currently no standardized quality-of-life measurement instruments.

The gambling utility analysis is performed by asking patients what percent risk of immediate death, if any, they would be willing to assume if the alternative is permanent normal health. The percent risk assumed is subtracted from 1.0 to arrive at the utility value.

The willingness-to-pay utility analysis is completed by asking patients what proportion of their monthly wage, or some other amount, if any, they would be willing to pay in return for permanent normal health. The proportion is subtracted from 1.0 to derive the utility value.

The time-tradeoff utility analysis is conducted by asking patients what proportion of their theoretically remaining time of life, if any, they would trade in return for permanent normal health. The proportion is subtracted from 1.0 to derive the utility value.

Continuous scaling instruments ask patients to choose a point estimate from 0 to 100 or 0.00 to 1.00 which they believe correlates with the quality of life associated with their health state. The point chosen by the patient is transformed to a corresponding on a scale from 0 to 1 to become the utility value.

Multiattribute instruments, such as EuroQol and the Health Utilities Index, ask question about dealing with certain quality-of-life aspects, such as mobility, self-care, usual activity, pain, anxiety, or discomfort. Patients assign a disutility value associated with each quality-of-life aspect. The disutility values are subtracted from 1.00 to derive utility values. EuroQol is described in the publication Health Policy under EuroQol: A New Facility for the Measurement of Health-Related Quality of Life by the EuroQol Group (1990). The Health Utilities Index and its variations are discussed in the publication Methods for the Economic Evaluation of Health Care Programmes, 2nd Edition by M. F. Drummond, B. O'Brien, G. L. Stoddart, and G. W. Torrance (Oxford University Press 2000).

A good health-related quality-of-life measurement instrument should be all-encompassing as to variables that compose quality of life, sensitive to small changes in health, reliable or reproducible, applicable to all medical specialties, able to be completed within a reasonable time period, readily understandable by patients, able to measure what it is intended to measure, and able to be integrated with healthcare costs for the performance of health-care economic analyses.

### SUMMARY OF THE INVENTION

Accordingly, it is one object of the invention to provide a standardized cost-utility analysis for pharmaceutical interventions since currently there are no standardized quality-of-life measurement instruments or standardized utility values. The lack of standards for cost-utility analysis prevents widespread acceptance of value-based medicine, and value-based medicine allows pharmaceutical dollars to be spent in the most efficient manner and facilitate the provision of pharmaceuticals to all patients in need. Value-based medicine achieved through cost-utility analysis also maximizes efficient use of healthcare resources by not only spending less money but also by deriving the greatest possible value for the money expended.

The invention uses patient perceived value, the utility value, and objective value, clinical trial data, of various pharmaceutical interventions to compare those pharmaceutical interventions. It uses standardized utility values to determine mean utility values for each pharmaceutical intervention. The mean utility value takes into account the benefits, side effects, and negative effects associated with each pharmaceutical intervention. It is the combination of the probability of each benefit, side effect, or negative effect occurring and the associated patient perception of each benefit, side effect, or negative effect. Then the mean utility value is combined with the probability of the pharmaceutical intervention improving health, and the combination of the probability of no improvement and its associated utility value to provide the final outcome utility value. The final outcome utility value is the most probable outcome of a pharmaceutical intervention. The difference in final outcome utility values between two pharmaceutical interventions presents the most probable gain. Then, by incorporating the cost for each pharmaceutical intervention, the gain per dollar expended for each pharmaceutical intervention is provided for comparison so that the most effective pharmaceutical intervention for the money expended can be chosen.

In one embodiment of the invention, a system for performing a cost-utility analysis of pharmaceutical interventions is provided. The system includes a processor and a database. The database contains for each pharmaceutical intervention several utility values associated with each health state and a probability for each potential health state associated with each pharmaceutical intervention. The processor is in communication with the database and determines a mean utility value for each pharmaceutical intervention by correlating each probability associated with each pharmaceutical intervention with the utility value associated with the respective health state. The processor also compares the pharmaceutical interventions the mean utility values of the pharmaceutical interventions by decision analysis.

In accordance with another embodiment of the invention, a method for performing a cost-utility analysis of a pharmaceutical intervention is disclosed. The first step is determining a mean utility value for each pharmaceutical intervention by correlating the probability of each health state associated with the pharmaceutical intervention with a utility value associated with the respective health state. The next step is comparing the mean utility values of each pharmaceutical intervention by decision analysis.

These and other objects of the invention, as well as many of the intended advantages thereof, will become more readily apparent when reference is made to the following description, taken in conjunction with the accompanying drawings.

### BRIEF DESCRIPTION OF THE FIGURES

FIG. 1 is a block diagram of a system for performing a cost-utility analysis for pharmaceutical interventions in accordance with the preferred embodiment of the invention.

FIG. 2 is a flow diagram showing operations performed by modules in the system.

FIG. 3 is an exemplary output provided by the system.

FIG. 4 is a flow diagram showing a method of performing a cost-utility analysis for pharmaceutical interventions in accordance with an embodiment of the invention.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose.

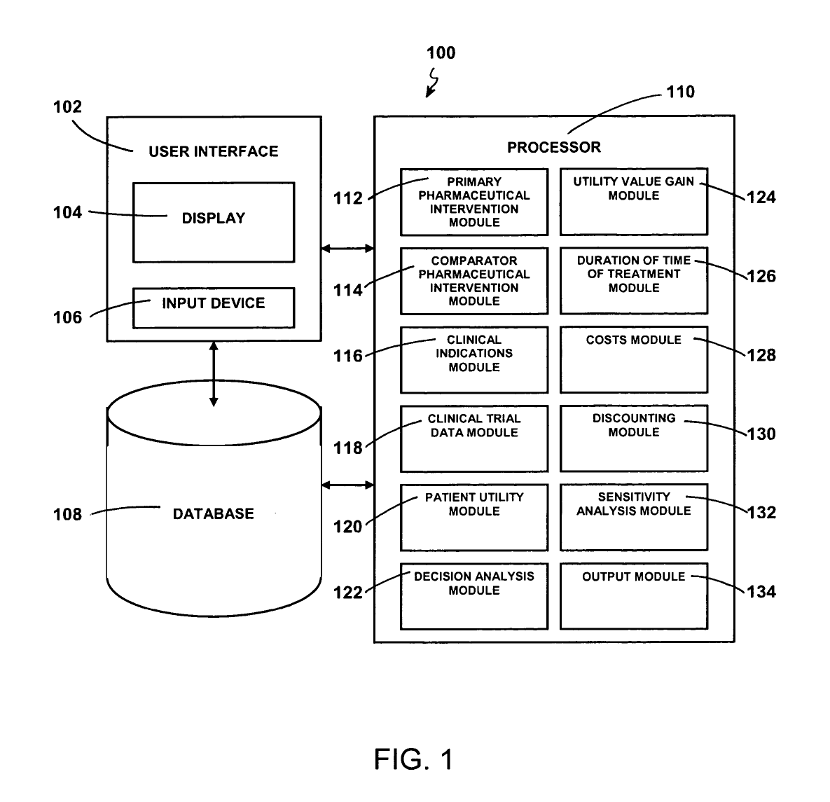

Turning to the drawings, FIG. 1 shows a system for performing a cost-utility analysis of pharmaceutical interventions. The system 100 generally comprises a user interface 102, a database 108, and a processor 110. The user interface 102, the database 108, and the processor 110 can each be coupled to the Internet or a network such as a local area network (LAN) or wide area network (WAN). The system is not limited to hard-wired connections but can include wireless communication such as radio frequency (RF), 802.11 (WiFi), Bluetooth or any combination of data communications paths known to one skilled in the relevant art. For example, the system can be implemented or incorporated as a single device such as a personal digital assistant (“PDA”) or the database 108 can be placed on a remote server coupled to the Internet by hard-wired connections with other components located nearby in wireless communication with the Internet.

The user interface 102 is in communication with the database 108 and the processor 110. The user interface 102 can be a desktop, handheld, and/or touchscreen computing device or any other display and information input device. It has a display 104 and an input device 106. The display 104 can be any device that presents information to the user. The input device 106 can be a keyboard, mouse, or another similar device. At the user interface, the user can view information about patient and pharmaceutical interventions or enter additional information, including a diagnosis of the patient's current health state and current treatment. The health state is the state of a person's health, and can range from death to perfect health. The health state includes one or more diseases and different degrees of severity of those diseases.

The database 108 is in communication with the user interface 102 and the processor 110. The database 108 stores information, such as patient data, current health state information, health states corresponding to a particular pharmaceutical intervention, data regarding comparable pharmaceutical interventions, clinical indications for pharmaceutical interventions, clinical trial data, utility values, utility value methodologies, data about respondents providing utility values, analysis perspective, costs for pharmaceutical interventions, cost bases, and an annual rate for discounting. Though a single database 108 is shown in the embodiment of FIG. 1, more than one database can be provided, in which case each separate database is preferably in communication with each other, the user interface 102, the processor 110, or any combination of these components.

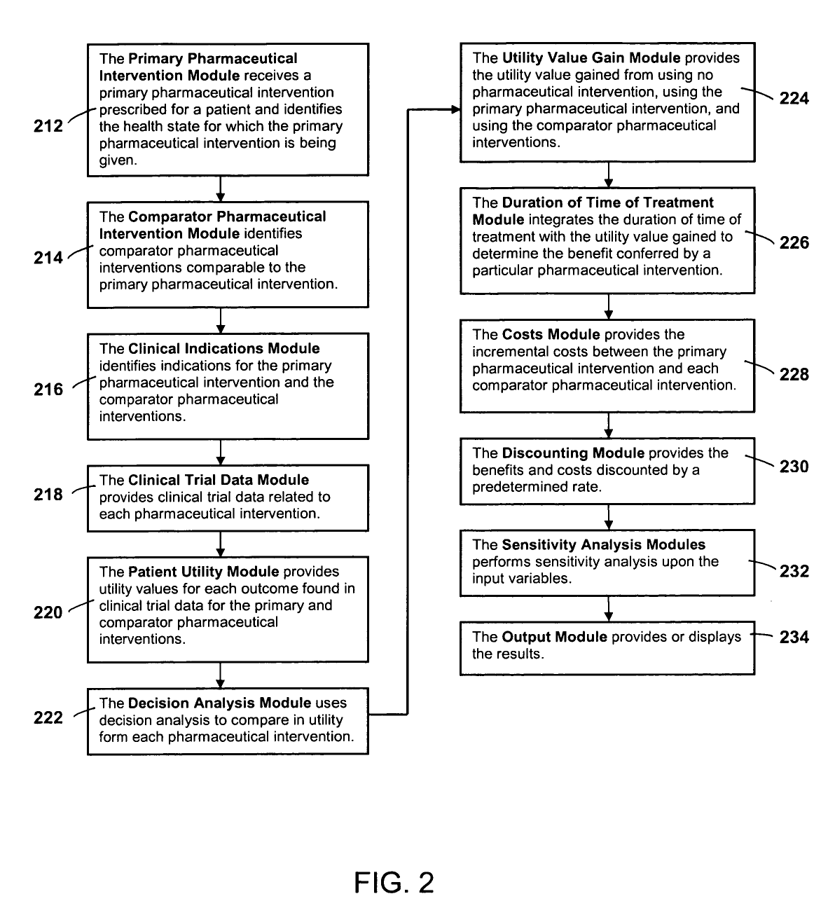

The processor 110 is in communication with the user interface 102 and the database 108. The processor preferably has one or more of the following modules: a primary pharmaceutical intervention module 112, a comparator pharmaceutical intervention module 114, a clinical indications module 116, a clinical trial data module 118, a patient utility module 120, a decision analysis module 122, a utility value gain module 124, a duration of time of treatment module 126, a costs module 128, a discounting module 130, a sensitivity analysis module 132, and an output module 134. As can be appreciated by one of ordinary skill in the art, each of the modules described herein can be implemented as one or more sub-routines, procedures, definitional statements, macros, or other similar processes. The description of each of the modules is used for convenience to describe the functionality of the system. Thus, the processes that are performed by each of the modules may be arbitrarily redistributed to one of the other modules, combined together in a single module, or made available in a shareable dynamic link library. FIG. 2 is a flow diagram showing the operations performed by the modules.

Referring to FIGS. 1 and 2, in step 212, the primary pharmaceutical module 112 receives the primary pharmaceutical intervention and identifies the health state for which it is prescribed. The primary pharmaceutical intervention module 112 is configured to receive the primary pharmaceutical intervention entered into the processor 110 from the user interface 102. Based on the entered primary pharmaceutical intervention, the primary pharmaceutical intervention module 112 identifies the health state for which the primary pharmaceutical intervention is being given by accessing information stored in the database 108. The health state can be determined according to, for example, a predefined algorithm or by referring to table that correlates the health state to the primary pharmaceutical intervention.

The primary pharmaceutical intervention and corresponding health state information are then used to identify comparable pharmaceutical interventions, step 214. Here, the comparator pharmaceutical intervention module 114 identifies comparator pharmaceutical interventions comparable to the primary pharmaceutical intervention. The comparator pharmaceutical intervention module 114 is configured to identify possible comparable pharmaceutical interventions based on the primary pharmaceutical intervention. Module 114 can also be configured to identify comparator pharmaceutical interventions by the health state, since the health state for which the primary pharmaceutical intervention is prescribed is identified by the primary pharmaceutical intervention module 112. The comparator pharmaceutical intervention module 114 accesses pharmaceutical intervention information stored in the database 108. The module 114 identifies the comparable pharmaceutical intervention by, for example, a predefined algorithm or use of tables. The comparator pharmaceutical interventions are identified so that a cost-utility comparison can be performed between comparable pharmaceutical interventions.

Then, in step 216, the clinical indications module 116 identifies indications for the primary pharmaceutical intervention and the comparator pharmaceutical interventions from steps 212 and 214, respectively. The clinical indications module 116 is configured to identify indications based on data stored in the database 108. An indication is a cause, an issue, a pathology, or a treatment of the particular health state or disease. The identification process can be performed by, for example, a predefined algorithm, use of tables, or other similar processes. Indications need to be identified so that comparable pharmaceutical interventions can be compared based on the same indications.

In step 218, the clinical trial data module 118 provides clinical trial data related to each pharmaceutical intervention, identified at step 216. The clinical trial data module 118 is configured to access clinical data from the database 108 related to each pharmaceutical intervention. Preferably, the clinical data is scientifically rigorous evidence-based clinical data that comes from randomized clinical trials and other peer-reviewed data as opposed to data derived from deductive reasoning supported by expert opinion. Expert opinion data can be flawed because compliance is not adequately controlled in clinical practice, patients who are lost in follow up interviews are not adequately considered, unusual results frequently regress toward the mean, randomization does not typically occur, and studies are often not double blind. The clinical data should preferably be based on Level 1 evidence which has low Type 1 and Type 2 error. Type 1 error is the chance of accepting a false-positive outcome and is preferably less than or equal to 0.05. Type 2 error is the chance of accepting a false-negative outcome and is preferably less than or equal to 0.20. The clinical data identifies possible outcomes for the primary pharmaceutical intervention and any comparator pharmaceutical interventions. Outcomes include benefits, side-effects, death, and other resulting health states. The clinical trial data module 118 provides the expected outcomes from using each pharmaceutical intervention. The outcomes can include adverse effects such as side effects, no effect, or beneficial effects on the health state of a patient. Each outcome is used in utility analysis to derive the utility value that incorporates all the outcomes of using a particular pharmaceutical intervention. Thus, the comparison of different pharmaceutical interventions considers all effects resulting from each pharmaceutical intervention.

In step 220, the patient utility module provides utility values for each outcome found in the clinical trial data of step 218 for the primary and comparator pharmaceutical interventions. The patient utility value module 120 retrieves the utility value associated with each outcome of the pharmaceutical interventions. From the database 108, the patient utility value module 120 obtains the relevant utility value corresponding to all expected outcomes found in the clinical trials data. The module 120 obtains relevant utility values by way of a predefined algorithm, use of tables, or other similar processes. The utility values are needed to convert the outcomes of each pharmaceutical intervention into utility analysis form. The conversion to utility analysis form is performed by the decision analysis module 122.

Utility values are obtained by asking a group of persons about their health state. Enough people should be asked about a particular health state so that the utility value derived for that health state can be statistically applied to another group of people with the same health state.

Utility values obtained from preference-based instruments are preferably used. Preference-based instruments measure the quality of life associated with a health state. Preference-based health-related quality-of-life measurement instruments require a subject to make a decision between his or her current health state and the alternative of trading or risking something of value, such as time of life, money, or life itself, for a return to perfect health. They also include rating scales and multivariable instruments.

Preferably, the health-related quality-of-life measurement instrument can be correlated with the International Classification of Diseases and Current Procedural Terminology (CPT) codes since both are utilized for healthcare intervention payment. The CPT codes are discussed in International Classification of Diseases, 9th Revision, Clinical Modifications by A. C. Hart and C. A. Hopkins (Ingenix 2003). For each CPT code, the corresponding utility value is preferably associated with the disease or other health state classified with that particular CPT code.

Also, quality-of-life instruments obtained using an interviewer is preferred to self-administered quality-of-life instruments. Telephone interviews can be used. Utility values obtained by telephone interview are similar to those obtained by face-to-face, interviewer-administered, utility values.

Preferably, the utility values from time-tradeoff utility analysis are used. Time-tradeoff utility values are applicable across all diseases, reliable or reproducible, valid measurements of what is intended to be measured, easily comprehensible by patients, and low administrative burdens. Time-tradeoff utility values are generally unaffected by age, ethnicity, gender, level of education, or income. Time-tradeoff utility values are applicable across virtually all segments of the population. Time-tradeoff utility values preferably range from a lower value of 0.0 corresponding to death to an upper value of 1.0 corresponding to permanent perfect health. When obtaining time-tradeoff utility values, preferably the exact disease under study, the severity of the disease, and treatment are clearly defined.

Utility values obtained from patients with the disease under study are preferred. Responses from treating physicians and other members of the medical community generally underestimate the decrease in quality of life caused by a disease as compared to patients who live or have lived with the disease. The validity of utility values obtained from children is uncertain. So, for diseases that affect both children and adults, utility values from affected adults are preferably used. For diseases that affect only children, proxy utility values obtained from adults who care for the children, such as parents, are preferably used.

Published utility values are also available, for instance, One Thousand Health-Related Quality-of-Life Estimates by T. 0. Teng and M. A. Wallace in Medical Care (2000) and Health Care Economic Analyses and Value-Based Medicine by M. M. Brown, G. C. Brown, S. Sharma, and J. Landy in Survey of Ophthalmology (2003). Many other publications provide utility values. Peer-reviewed literature is available at www.ncbi.nlm.nih.gov which contains the abstracts of over 15 million articles at the National Library of Medicine.

Once relevant utility values corresponding to all expected outcomes based on clinical trials are obtained from the database 108 and each utility value is correlated with each corresponding outcome, a decision analysis to compare in utility form each pharmaceutical intervention is performed in step 222. The decision analysis module 122 performs the decision analysis. First, the utility values are used to convert the outcomes of each pharmaceutical intervention into utility form. Preferably, the utility form is the probability of each outcome occurring weighed by the utility value for that particular outcome of the pharmaceutical intervention. Once outcomes are converted into utility form, they are applied in the decision analysis. The decision analysis determines the most probable outcome of an intervention. It compares the most probable outcome of one intervention with the most probable outcome of another intervention or with no intervention at all. Thus, the decision analysis provides the optimal treatment option when deciding which intervention to prescribe.

The decision analysis is performed through deterministic modeling, stochastic modeling, or a combination of both as described in the publication Primer on Medical Decision Analysis: Part 3—Estimating Probabilities and Utilities by G. Naglie, M. D. Krahn, D. Naimark, D. A. Redelmeier, and A. S. Detsky in Medical Decision Making (1997). A deterministic model calculates mathematically the expected value for each pharmaceutical intervention. A stochastic model is a simulation that is performed a large number of times to provide statistical information for each pharmaceutical intervention.

Deterministic modeling is preferably used. In particular, simple decision analysis is preferably used where the most probable outcome of a pharmaceutical intervention is determined using decision tree models. Decision tree models are a sequence of decisions and events over time where every event is assigned a probability. Decision trees are discussed in Primer on Medical Decision Analysis: Part 1—Getting Started by A. S. Detsky, G. Naglie, M. D. Krahn, and D. Naimark in Medical Decision Making (1997); Primer on Medical Decision Analysis: Part 2—Building a Tree by A. S. Detsky, G. Naglie, M. D. Krahn, and D. Naimark in Medical Decision Making (1997); and Decision Analysis by S. G. Pauker and J. P. Kassirer in the New England Journal of Medicine (1997). The decision can be whether or not to use the pharmaceutical intervention, and the events can be the expected outcomes based on clinical data. Several paths are possible through the decision tree because each path can encompass several different decisions and events. Each decision alternative is evaluated with utility values weighted by the probability of the outcome. The decision alternative with the largest expected utility is the preferred decision.

Markov modeling and Monte Carlo simulation are adjuncts to decision analysis that can be employed to determine the expected value for a particular pharmaceutical intervention. Markov modeling is described, for instance, in the following publications: Chapter entitled “Markov Models in Medical Decision Making: A Practical Guide” by F. A. Sonnenberg and J. R. Beck in Medical Decision Making (1993); Meta-Analysis, Decision Analysis and Cost Effectiveness by D. B. Pettiti (Oxford University Press 2000); The Cost-Effectiveness of Photodynamic Therapy for Fellow Eyes with Subfoveal Choroidal Neovascularization Secondary to Age-Related Macular Degeneration by S. Sharma, G. C. Brown, M. M. Brown, H. Hollands, and G. K. Shah in Ophthalmology (2001); and DATA 4.0 Healthcare User's Manual by TreeAge Software, Inc. (TreeAge Software, Inc. 2003). Monte Carlo modeling is discussed, for instance, in the publications: Incremental Cost-Effectiveness of Therapeutic Interventions for Branch Retinal Vein Occlusion by G. C. Brown, M. M. Brown, S. Sharma, B. Busbee, and H. Brown in Ophthalmic Epidemiology (2002); A Cost-Utility Analysis of Interventions for Proliferative Vitreoretinopathy by G. C. Brown, M. M. Brown, S. Sharma, and B. Busbee in the American Journal of Ophthalmology (2002); and A Cost-Utility Analysis of Laser Photocoagulation for Extrafoveal Choroidal Neovascularization by B. Busbee, M. M. Brown, G. C. Brown, and S. Sharma in Retina (2003). Markov modeling analysis measures the recurrent risk of an event and can be used in cases with recurrent outcomes, such as treatment for hypertension. Even with treatment, the chance of cardiac death that occurs each year must be accounted for in decision analysis. The recurrent risk can only be accounted for in the first year by simple decision analysis. Monte Carlo simulation is a stochastic model that uses a reference-case, such as the average person, to perform a hypothetical trial with a particular pharmaceutical intervention. The model is run several times and the outcome changes each time because of chance events occurring so that it can calculate the range, mean, median, and 95% confidence interval for a particular pharmaceutical intervention. When decision analysis is complete, utility values adjusted for expected outcomes are provided.

In the preferred embodiment using simple decision analysis, each outcome such as benefits, side effects, death, and other health states, is multiplied by the utility value for that particular outcome to convert each outcome into utility form. The probability of no adverse effects is also multiplied with its associated utility value. Then the results are summed to obtain a mean utility value which incorporates all expected outcomes for the particular pharmaceutical intervention. Specifically, the mean utility value is (probability of health state 1)*(utility value of health state 1)+(probability of health state 2)*(utility value of health state 2)+ . . . +(probability of health state n)*(utility value of health state n), where n represents the number of different possible health states as determined by clinical trial data.

Then, using the decision tree, the probability for the final outcome of using the pharmaceutical intervention, such as disease treated or patient cured, is multiplied with the associated mean utility value for that particular pharmaceutical intervention. The probability of the final outcome not occurring, such as disease is not treated or the patient is not cured, is multiplied with the associated utility value for the pharmaceutical intervention failing to provide the final outcome. The sum of the two calculations provides the final outcome utility value. Similar determinations for other pharmaceutical interventions are performed. Further, the probability of the final outcome occurring with no pharmaceutical intervention is multiplied with its associated utility value. The probability of the final outcome not occurring is also multiplied with its associated utility value. Summing the two results provides the final outcome utility value for no pharmaceutical intervention.

Predetermined decision analysis from deterministic modeling, stochastic modeling, or a combination of both can be stored in the database 108. The pharmaceutical intervention decision analysis module 122 then completes the decision analysis by accessing the predetermined decision analysis stored in the database 108 and performing the predetermined decision analysis, using predefined algorithms based on decision analysis modeling, using tables, or other similar processes. Regardless of the particular process, the decision analysis module 122 provides the most probable outcome of a pharmaceutical intervention and therefore the optimal treatment option when deciding which intervention to prescribe.

In step 224, the utility value gain module 124 provides the utility gained from using no pharmaceutical intervention, using the primary pharmaceutical intervention, and using the comparator pharmaceutical intervention. The utility value gain module 124 is configured to compare the results of prescribing no pharmaceutical intervention, prescribing the primary pharmaceutical intervention, and prescribing the comparator pharmaceutical intervention. The module 124 determines the difference between the final outcome utility value for the primary pharmaceutical intervention and the final outcome utility value for no pharmaceutical intervention. The module 124 completes similar determinations for each comparator pharmaceutical intervention. The difference between final outcome utility values provides the utility value gained.

Next, in step 226, the duration of time of treatment module 126 integrates the duration of time of treatment with the utility value gained to determine the benefit conferred by a particular pharmaceutical intervention. The duration of time of treatment module 126 is configured to calculate the benefit conferred by each pharmaceutical intervention. Preferably, it integrates the duration of treatment benefit, preferably in years, with the difference in final outcome utility values between two pharmaceutical interventions to obtain the total quality gain. The module 125 can also calculate the percent improvement in the length of life, the quality of life, or both conferred by the pharmaceutical intervention. Preferably, a quality-adjusted life-year (“QALY”) is used. The QALY is a measure of life's value accrued over time. For example, living at a utility value of 1.0 for one year accrues one QALY, while living at a utility value of 0.5 for one year accrues 0.5 QALY. The QALY incorporates all improvements in length of life, quality of life, or both conferred by the pharmaceutical intervention. So, the QALY's conferred by the pharmaceutical intervention objectively measure the total value gained from the intervention and can also integrate all adverse effects induced by healthcare intervention. QALY's are comparable across all interventions in healthcare.

Then, in step 228, the costs module 128 provides the incremental costs between the primary pharmaceutical intervention and each comparator pharmaceutical intervention. The costs module 128 provides the difference in costs between using the primary pharmaceutical intervention and one of the comparator pharmaceutical interventions. The difference includes those costs incurred or saved as a result of the pharmaceutical intervention, without which, they would not have occurred. The difference in costs is preferably measured in dollars expended per quality-adjusted life-year or $/QALY. This determination can be performed according to, for example, a predefined algorithm or by using tables. The difference in costs between pharmaceutical interventions is determined so that one pharmaceutical intervention can be readily compared in terms of costs with another pharmaceutical intervention.

Costs can include direct healthcare costs, direct nonhealthcare costs, and indirect healthcare costs. Direct healthcare costs are those associated with goods, services, and other resources that are consumed in the provision of an intervention or in dealing with the side effects or other current and future consequences linked to the intervention. Direct healthcare costs can include physician service costs, acute hospital costs, ambulatory surgery centers costs, skilled nursing facility costs, rehabilitation costs, nursing home costs, home health care costs, pharmaceutical costs, clinical test costs, diagnostic study costs, durable goods costs, and other similar costs. Direct nonhealthcare costs can include care provided by friends and family, transportation costs, childcare costs, housekeeping costs, retaining costs, and other similar costs. Indirect healthcare costs or productivity costs can include lost patient wages, lost patient nonwork time, lost tax revenue, lost productivity from premature death, disability payment costs, and other similar costs. Preferably, only direct healthcare costs are used. In particular, pharmaceutical costs are preferably calculated using the average wholesale price.

Next, in step 230, the discounting module 130 provides the benefits and costs discounted by a predetermined rate. The discounting module 130 is preferably configured to discount the QALY's and costs by an annual rate. Discounting is used to account for the time value of money and the time value of the results of pharmaceutical intervention. Discounting accounts for inflation which reduces the value of money as time passes. A net present value analysis of healthcare intervention discounts future costs and value gained to their present value. Both costs and results should be discounted at the same rate. The Panel for Cost-Effectiveness in Health and Medicine recommends a 3% annual discount rate for healthcare costs and outcomes. The annual rate can be the recorded rate of inflation, expected rate of inflation, or any other suitable rate. The discounting can be performed according to, for example, a predefined algorithm or by using tables.

In step 232, the sensitivity analysis module 132 performs sensitivity analysis upon the input variables. The sensitivity analysis module 132 is configured to perform sensitivity analysis on any of the inputs, such as utility value, clinical trial data, costs, or any other data used by the system 100. Sensitivity analysis should be performed on data with the lowest level of confidence or have the greatest impact on the analysis. One-, two-, three-, or n-way sensitivity analyses can be performed by varying one or more parameters in the decision analysis. One variable is changed at a time or multiple variables are changed simultaneously. One-way sensitivity analysis is preferably performed on as many variables as possible and n-way sensitivity analysis is preferably performed on parameters that have a large degree of uncertainty or are highly influential. Also, sensitivity analysis is preferably performed for economic evaluations. In particular, it is performed by varying the discount rate of 3% to between 0 and 5% to ascertain the effect of discounting and to allow better comparability. The sensitivity analysis can be performed according to, for example, a predefined algorithm or by using tables.

Finally, in step 234, the output module 134 provides or displays the results. The output module 134 is configured to provide an output to allow the user to choose either the primary pharmaceutical intervention or one of the comparator pharmaceutical interventions that can provide equal patient value for less cost. The output module 134 preferably provides a Pharmaceutical Value Index Report.

FIG. 2 shows an exemplary Pharmaceutical Value Index Report that is generated by the output module 134. The report provides the primary pharmaceutical interventions 312, comparator pharmaceutical interventions 314, and clinical indication 316 in the upper part of the report. Additional information such as the drug class 318 for the primary pharmaceutical intervention can also be provided. In the middle of the report, for each pharmaceutical intervention, the utility value gain 324 is provided in the first column after the list of pharmaceutical interventions. The utility value gains are also provided in percentage form 325 in the next column. Costs 328 for one year's treatment are provided for each pharmaceutical intervention. Costs relative to the cheapest pharmaceutical intervention 329 are listed in the adjacent column. Finally, the difference in costs measured in dollars expended per quality-adjusted life-year or $/QALY 330 is in the last column. Sensitivity analyses results 332 are annotated below the columns. A summary 334 of the results is provided near the bottom of the report.

The output can be used by prescribing medical professionals, patients, managed care organizations, health insurers, pharmacy benefit managers, pharmacists, state and federal organizations, self-insured companies, labor unions, and other healthcare stakeholders. The output can be used to establish preferred drug lists based on value-based medicine. Also, it can be used to place drugs into tiers such as tiers based upon comparable drug value, drugs with similar co-payments, or drugs that require pre-authorization. The output allows entities that purchase pharmaceuticals to more effectively negotiate pricing with drug manufacturers since the respective value of the drugs are known and less expensive alternatives identified.

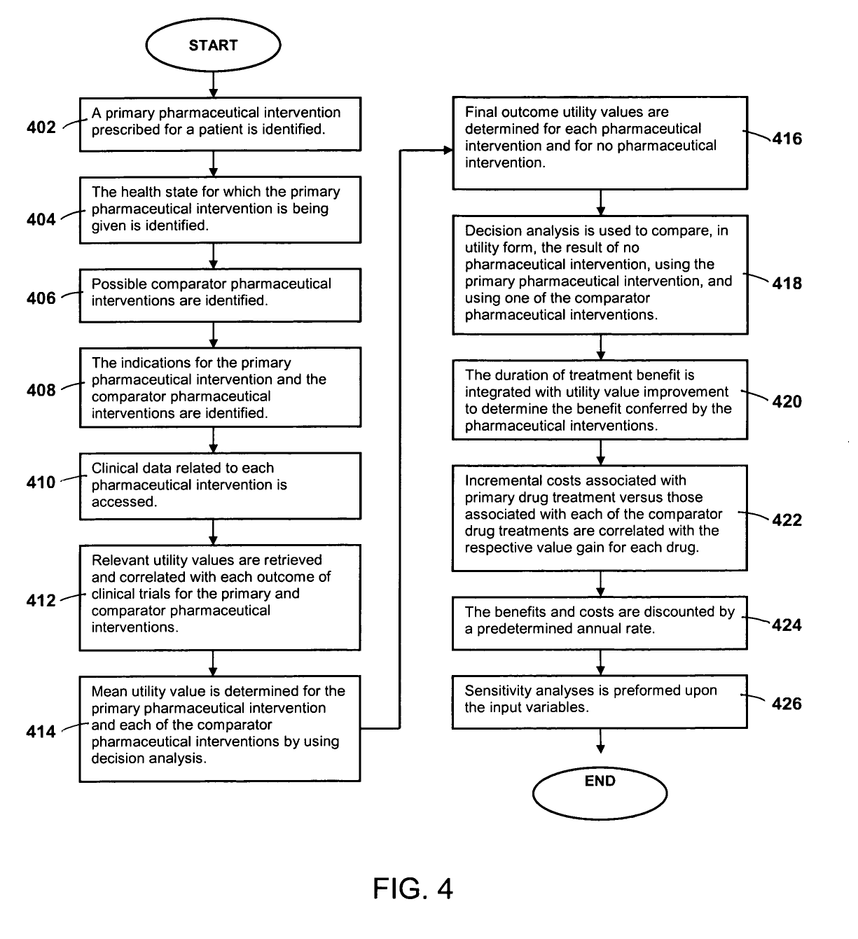

FIG. 4 is a flow chart showing a method of performing a cost-utility analysis for pharmaceutical interventions. Depending on the embodiment, additional steps may be added, others removed, and the order of the steps arranged. In step 402, a primary pharmaceutical intervention prescribed for a patient is identified. Next, the health state for which the primary pharmaceutical intervention is being used is identified, step 404. Then in step 406, at least one comparator pharmaceutical intervention for the primary pharmaceutical intervention is identified. Clinical trials related to the primary pharmaceutical intervention and each of the comparator pharmaceutical interventions are identified in step 410. In step 412, each outcome of the clinical trials is assigned a utility value. Then, a mean utility value is calculated for the primary pharmaceutical intervention and each of the comparator pharmaceutical interventions by using decision analysis, step 414.

Afterwards, in step 416, final outcome utility values are determined for each pharmaceutical intervention and for no pharmaceutical intervention. Then, in step 418, decision analysis is used to compare, in utility form, the result of no pharmaceutical intervention, using the primary pharmaceutical intervention, and using one of the comparator pharmaceutical interventions. The utility value improvement from treatment versus no treatment is ascertained for the primary pharmaceutical intervention and comparator pharmaceutical interventions.

In step 420, the duration of treatment benefit is integrated with the utility value improvement conferred by the primary and comparator pharmaceutical interventions. Preferably, the duration of treatment benefit, in years, is used to calculate the total value, or number of quality-adjusted life years (QALYs), conferred by the primary and comparator pharmaceutical interventions. The QALY gain is calculated by multiplying the utility value improvement from treatment with the duration of treatment benefit, in years or QALY=(utility value improvement from treatment)*( duration of treatment benefit, in years). An improvement in value conferred can also be provided in percent form. Also, the improvement in value conferred in the form of length of life gain can be calculated by multiplying the years of life gain by the utility value at which the patients live during the extra years of life or (years of life gain)*(utility value at which the patients live during the extra years of life).

Next, in step 422, incremental costs associated with the primary pharmaceutical intervention versus those associated with each of the comparator pharmaceutical interventions are correlated with their respective value gains. The output is preferably in dollars expended per quality-adjusted life-year ($/QALY).

Additionally, in step 424, value gained or QALY and costs are discounted by an annual rate to calculate a present value. The annual rate is preferably determined based on economic conditions.

Finally, in step 426, sensitivity analysis can be performed on the input variables, such as utility values, clinical trial data, and costs. Sensitivity analysis should be performed on input variables about which there is the least confidence or have the greatest influence on the analysis.

The calculations completed by the method can be performed according to, for example, a predefined algorithm or by using tables. The present invention may be implemented with any combination of hardware and software. If implemented as a computer-implemented apparatus, the present invention is implemented using means for performing all of the steps and functions described above.

The following example is provided to illustrate a cost-utility analysis for pharmaceutical interventions in accordance with the invention, but is not intended to be limiting to the invention. A patient is prescribed Aciphex, and Aciphex is entered into the primary pharmaceutical intervention module 112, step 402. The clinical indications module 116 identifies Aciphex as being used for gastroduodenal ulcer, step 404. The comparator pharmaceutical intervention module 114 then identifies a comparator pharmaceutical intervention, Omeprazole, step 406. At step 410, the clinical trial data module 118 accesses the clinical trial data related to both Aciphex and Omeprazole from database 108. The patient utility module 120 retrieves utility values from the database 108 and correlates relevant utility values to each outcome found in clinical trials for Aciphex and Omeprazole, step 412. At step 414, according to the clinical data, the system determines that Aciphex had the adverse effects of heartburn in 20% of the cases, nausea in 10% of the cases, and rash in 10% of the cases. No adverse effects were shown in 60% of the cases. The utility values associated with heartburn, nausea, rash, and no adverse effects are 0.92, 0.80, 0.90, and 1.00, respectively. Thus, the decision analysis module 122 determines that the mean utility value is (0.20)*(0.92)+(0.10)*(0.80)+(0.10)*(0.90)+(0.60)*(1.00) or 0.954. Similar analysis for Omeprazole according to its adverse effects and utility values results in 0.961 as its mean utility value.

Next, the decision analysis module 122, at step 416, determines the final outcome utility value for each pharmaceutical intervention and for no pharmaceutical intervention. With no treatment, the patient has a 30% chance that the ulcer will go into remission with an associated utility value of 1.00 and a 70% chance of no remission which has a utility value of 0.75. Thus, the decision analysis module 122 determines that the final outcome utility value is (0.30)*(1.00)+(0.70)*(0.75) or 0.825. With Aciphex, the patient has a 70% chance of the ulcer going into remission with an associated mean utility value of 0.954 (calculated previously to take into account the adverse effects of Aciphex) and a 30% chance of no remission which has a utility value of 0.75. So, the final outcome utility value for Aciphex is (0.70)*(0.954)+(0.30)*(0.75) or 0.893. And, with Omeprazole, the patient has a 72% chance of the ulcer going into remission with an associated mean utility value of 0.961 and a 28% chance of no remission which has a utility value of 0.75. The final outcome utility value for Omeprazole is then (0.72)*(0.961)+(0.28)*(0.75) or 0.902.

To complete the comparison, the utility value gain module 124 determines the utility value gain conferred by each drug over no treatment. The module 124 in accordance with step 418 finds the utility value gain for prescribing Aciphex and prescribing Omeprazole over prescribing no drugs. For Aciphex, the utility value gain is 0.893−0.825 or 0.068. For Omeprazole, the utility value gain is 0.902−0.825 or 0.077.

Then, the duration of time of treatment module 126 integrates the duration of treatment benefit in years to calculate the number of QALY's conferred by the drugs, and thus the module 126 performs step 420. The number of QALY's gained is (utility value gain conferred)*(time of benefit in years). For Aciphex, the QALY's gained are (0.068)*(1 year) or 0.068 QALY. For Omeprazole, it is (0.077)*(1 year) or 0.077 QALY. A percent improvement in the value of life can also be calculated. For Aciphex, the percent improvement in the value of life is (0.893−0.825)/0.825 or 8.2% improvement. For Omeprazole, it is (0.902−0.8250)/0.825 or 9.3%.

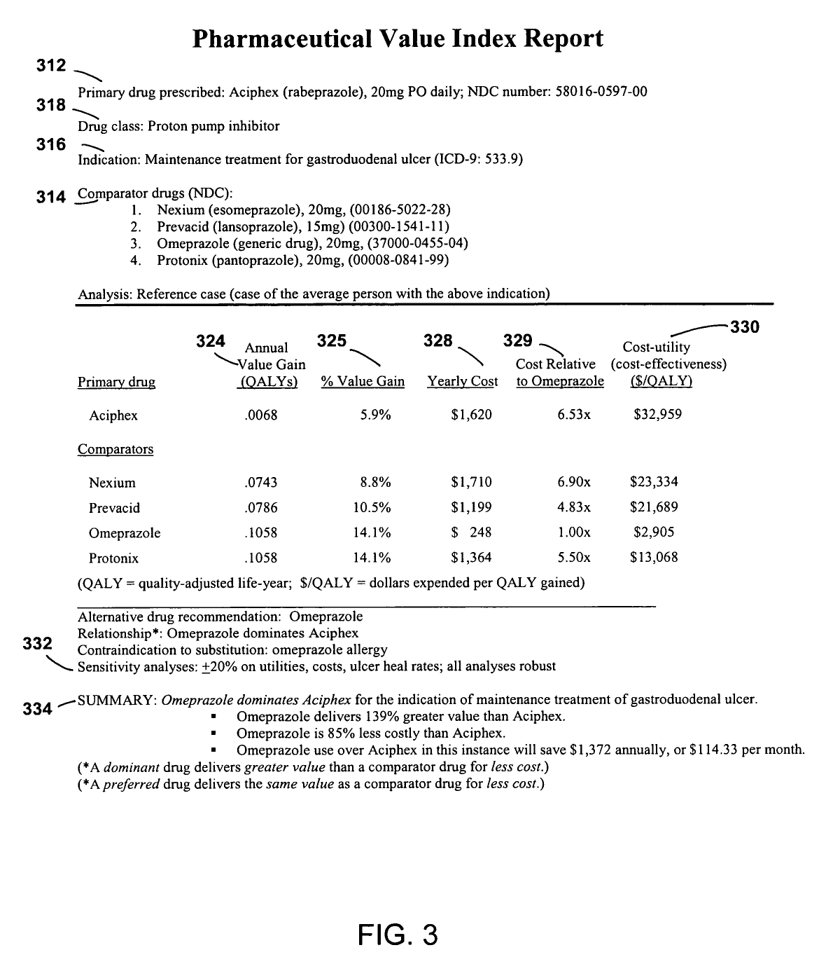

In FIG. 3, on the Pharmaceutical Value Index Report, the primary pharmaceutical intervention 312, Aciphex, is displayed. Omeprazole is listed as one of several comparator pharmaceutical interventions 314. The QALY's are listed as the annual value gain 324. Percent improvements are listed under percent value gain 325.

The duration of time of treatment module 126 can also correlate the value gain to the costs of each drug. The result can be expressed as dollars expended per QALY or $/QALY. If Aciphex costs $1,620 per year, then the $/QALY is $1,620/0.068 QALY or $23,800/QALY. If Omeprazole costs $248 per year, then the $/QALY is $248/0.077 QALY or $3,220/QALY. In FIG. 3, the results are listed as Cost-Utility (cost-effectiveness) ($/QALY) 330.

The present invention can be included in an article of manufacture (e.g., one or more computer program products) having, for instance, computer usable media. The media has embodied therein, for instance, computer readable program code means for providing and facilitating the mechanisms of the present invention. The article of manufacture can be included as part of a computer system or available separately.

The foregoing description and drawings should be considered as illustrative only of the principles of the invention. The invention may be configured in a variety of embodiments and is not intended to be limited by the preferred embodiment. Numerous applications of the invention will readily occur to those skilled in the art. Therefore, it is not desired to limit the invention to the specific examples disclosed or the exact operation shown and described. Rather, all suitable modifications and equivalents may be resorted to, falling within the scope of the invention.

We claim:

1. A system for performing a cost-utility analysis of pharmaceutical interventions, each pharmaceutical intervention associated with one or more potential health states, the system comprising:
a database containing for each pharmaceutical intervention a plurality of utility values associated with each respective health state, and a probability for each of the potential health states associated with each pharmaceutical intervention; and
a processor in communication with said database, wherein said processor determines a mean utility value for each pharmaceutical intervention by correlating each probability associated with each pharmaceutical intervention with the utility value associated with the respective health state; and said processor compares the mean utility values of the pharmaceutical interventions by decision analysis.

2. The system of claim 1, wherein the health state is the state of a person's health such as death, affliction by one or more diseases, or permanent perfect health.

3. The system of claim 1, wherein the utility value varies with the health state between 0.0 representing death and 1.0 representing permanent perfect health.

4. The system of claim 1, wherein the processor receives a primary pharmaceutical intervention and identifies a comparator pharmaceutical intervention.

5. The system of claim 1, wherein the processor determines a final outcome utility value for each pharmaceutical intervention by correlating a probability of improved health state with the mean utility value of the pharmaceutical intervention and integrating a correlation of a probability of no improvement in health state with its associated utility value, and the processor compares final outcome utility values of the pharmaceutical interventions.

6. The system of claim 5, wherein the processor determines a value gained by finding the difference between the final outcome utility values for the pharmaceutical interventions and the final outcome utility value for no pharmaceutical intervention.

7. The system of claim 6, wherein the processor integrates the valued gained with the duration of treatment benefit to determine the benefit conferred by the pharmaceutical intervention.

8. The system of claim 7, wherein the processor integrates the value gained with a year to obtain the quality-adjusted life-year or QALY.

9. The system of claim 1, wherein the processor performs sensitivity analysis.

10. A method for performing a cost-utility analysis of a pharmaceutical intervention, comprising:
determining a mean utility value for each pharmaceutical intervention by correlating the probability of each health state associated with the pharmaceutical intervention with a utility value associated with the respective health state; and comparing the mean utility values of each pharmaceutical intervention by decision analysis.

11. The method of claim 10, further comprising the step of identifying the health state associated with the pharmaceutical intervention.

12. The method of claim 10, further comprising the step of identifying a comparator pharmaceutical intervention.

13. The method of claim 10, further comprising the steps of:
determining a final outcome utility value for each pharmaceutical intervention by correlating a probability of improved health state with the mean utility value of the pharmaceutical intervention and integrating a correlation of a probability of no improvement in health state with its associated utility value;
determining a final outcome utility value for no pharmaceutical intervention by correlating a probability of improved health state with its associated utility value and integrating a correlation of a probability of no improvement in health state with its associated utility value.

14. The method of claim 13, further comprising the step of determining a value gained based on the final outcome utility values for the pharmaceutical intervention and the final outcome utility value for no pharmaceutical intervention.

15. The method of claim 14, further comprising the step of integrating the valued gained with the duration of treatment benefit to determine the benefit conferred by the pharmaceutical intervention.

16. The method of claim 10, wherein the value gained is integrated with one year to obtain the quality-adjusted life-year or QALY.

17. The method of claim 10, further comprising the step of performing sensitivity analysis.

18. The method of claim 10, wherein the health state is the state of a person's health such as death, affliction by one or more diseases, or permanent perfect health.

19. The method of claim 10, wherein the utility value varies with the health state between 0.0 representing death and 1.0 representing permanent perfect health.
